SEQUENCE LISTING (34 sequences)

SEQ ID NO: 1 - DNA, 21 bp, Artificial Sequence
  Primer pair sequence parts
gtttttcggagaaatcttcgg

SEQ ID NO: 2 - DNA, 21 bp, Artificial Sequence
  Primer pair sequence part
caaatgctggaatgtttccac

SEQ ID NO: 3 - DNA, 21 bp, Artificial Sequence
  Primer pair sequence part
aagcattgaatctggtggttg

SEQ ID NO: 4 - DNA, 21 bp, Artificial Sequence
  Primer pair sequence part
ttggtgtggttaggactttgg

SEQ ID NO: 5 - DNA, 23 bp, Artificial Sequence
  Primer pair sequence part
atgatgcagacttgttgtatcca

SEQ ID NO: 6 - DNA, 22 bp, Artificial Sequence
  Primer pair sequence part
cacagttcttcaaagaatctcc

SEQ ID NO: 7 - DNA, 54 bp, Artificial Sequence
  Primer pair sequence part
ggggacaagtttgtacaaaaaagcaggcttcatgaagaagcgcttaaccacttc

SEQ ID NO: 8 - DNA, 54 bp, Artificial Sequence
  Primer pair sequence part
ggggaccactttgtacaagaaagctgggtcttattcagaaccaacgaacaagcc

SEQ ID NO: 9 - DNA, 53 bp, Artificial Sequence
  Primer pair sequence part
ggggacaagtttgtacaaaaaagcaggcttcatggcgattttggattctgctg

SEQ ID NO: 10 - DNA, 55 bp, Artificial Sequence
  Primer pair sequence part
ggggaccactttgtacaagaaagctgggtctgacatcgatccttttcggttcatc

SEQ ID NO: 11 - DNA, 51 bp, Artificial Sequence
  Primer pair sequence part - Gene cloning
ggggacaagtttgtacaaaaaagcaggcttcatggcggatacagctagagg

SEQ ID NO: 12 - DNA, 48 bp, Artificial Sequence
  Primer pair sequence part - Gene cloning
gggaccactttgtacaagaaagctgggtcagtagtgtgctggccacca

SEQ ID NO: 13 - DNA, 21 bp, Artificial Sequence
  Primer pair sequence - qRT-PCR
ggaagtcagctaaggatggtg

SEQ ID NO: 14 - DNA, 21 bp, Artificial Sequence
  Primer pair sequende - qRT-PCR
gtccgaaaccaaacaagacac

SEQ ID NO: 15 - DNA, 21 bp, Artificial Sequence
  Primer pair sequence - qRT-PCR
aagagcttatggaacacctcg

SEQ ID NO: 16 - DNA, 20 bp, Artificial Sequence
  Primer pair sequence part - qRT-PCR
agacctttgttcacaccctg

SEQ ID NO: 17 - DNA, 20 bp, Artificial Sequence
  Primer pair sequence part - qRT-PCR
tcggaagagtgtttgggaac

SEQ ID NO: 18 - DNA, 21 bp, Artificial Sequence
  Primer pair sequence part - qRT-PCR
caatcgcagccatgtcatatg

SEQ ID NO: 19 - DNA, 20 bp, Artificial Sequence
  Primer pair sequence part - qRT-PCR
ccgacgcaatcttcaaacag

SEQ ID NO: 20 - DNA, 20 bp, Artificial Sequence
  Primer pair sequence part - qRT-PCR
ccgacgcaatcttcaaacag

SEQ ID NO: 21 - DNA, 20 bp, Artificial Sequence
  Primer pair sequence part - qRT-PCR
tgttgcagggcttttctctg

SEQ ID NO: 22 - DNA, 18 bp, Artificial Sequence
  Primer pair sequence part - qRT-PCR
tgttgagtcccatgtgcg

SEQ ID NO: 23 - DNA, 22 bp, Artificial Sequence
  Primer pair sequence part - qRT-PCR
atgacgcagagatggaacttag

SEQ ID NO: 24 - DNA, 21 bp, Artificial Sequence
  Primer pair sequence part - qRT-PCR
ggtgtcggcattcaaatactg

SEQ ID NO: 25 - DNA, 19 bp, Artificial Sequence
  Primer pair sequence part - qRT-PCR
agaactccgttgatgcctg

SEQ ID NO: 26 - DNA, 22 bp, Artificial Sequence
  Primer pair sequence part - qRT-PCR
acaattggtcctgctatggtac

SEQ ID NO: 27 - DNA, 21 bp, Artificial Sequence
  Primer pair sequence part - qRT-PCR
cgtcccatatgcgttgatttc

SEQ ID NO: 28 - DNA, 19 bp, Artificial Sequence
  Primer pair sequence part - qRT-PCR
tcccacctccaaacagttg

SEQ ID NO: 29 - DNA, 20 bp, Artificial Sequence
  Primer pair sequence part - qRT-PCR
aacagcaaaaccaacatccg

SEQ ID NO: 30 - DNA, 23 bp, Artificial Sequence
  Primer pair sequence part - qRT-PCR
cccttctgtaccttatctccaac

SEQ ID NO: 31 - DNA, 22 bp, Artificial Sequence
  Primer pair sequence part - qRT-PCR
cacaattaacagcacctccaag

SEQ ID NO: 32 - DNA, 21 bp, Artificial Sequence
  Primer pair sequence part - qRT-PCR
tgggataaagcaagagatggg

SEQ ID NO: 33 - DNA, 22 bp, Artificial Sequence
  Primer pair sequence part - qRT-PCR
ctccatacctaacttgcactcc

SEQ ID NO: 34 - DNA, 20 bp, Artificial Sequence
  Primer pair sequence part - qRT-PCR
ctctgcaccaagatcaccta

(19) United States
(12) Patent Application Publication
(10) Pub. No.: US 2019/0144880 A1
(43) Pub. Date: May 16, 2019

(54) Plants with Increased Oil Accumulation in Vegetative Tissues, and Methods of Making Same

(71) Applicant: Brookhaven Science Associates, LLC, Upton, NY (US)

(72) Inventors: John Shanklin, Shoreham, NY (US); Zhiyang Zhai, Middle Island, NY (US)

(21) Appl. No.: 16/100,323

(22) Filed: Aug. 10, 2018

Related U.S. Application Data
(60) Provisional application No. 62/544,349, filed on Aug. 11, 2017.

(51) Int. Cl. C12N 15/82

(52) U.S. Cl. CPC C12N 15/8246; A01H 6/20; C12N 15/8247

(57) ABSTRACT

The invention provides combining certain genetic mutations in plants to increase sugar levels in the vegetative tissues of the plants. The specific combinations of mutations yield increased oil production in the vegetative tissues. In one embodiment, a plant comprises two mutations, wherein the two mutations are an adg1 mutation and a suc2 mutation. In one embodiment, a plant comprises three mutations, wherein the three mutations are an adg1 mutation, a suc2 mutation and a sdp1 mutation, or comprises an adg1 mutation, a suc2 mutation and a tt4 mutation. In one embodiment, the plant comprises four mutations, wherein the four mutations are an adg1 mutation, a suc2 mutation, an sdp1 mutation, and a tgd1 mutation, or comprises an adg1 mutation, a suc2 mutation, a tt4 mutation and a tgd1 mutation.

### STATEMENT OF GOVERNMENT RIGHTS

The present application was made with government support under contract number DE-SC0012704 awarded by the U.S. Department of Energy. The United States government has certain rights in the invention(s).

### FIELD OF THE INVENTION

This application relates to sugar potentiation, fatty acid and triacylglycerol accumulation in vegetative tissues of plants, for example, in leaf tissue, and specifically sugar potentiation of fatty acid triacylglycerol accumulation in plants or vegetative plant tissues.

### BACKGROUND OF THE INVENTION

Sugars are the energy currency of the cell, fueling growth and development. Accordingly, the cell has a capacity to sense its sugar or energy status and to maintain a certain physiological status. When sugar and energy are above a certain level, anabolic metabolism predominates over catabolism to enable growth and development of the cell. Conversely, when sugar and energy are less than a certain level, growth slows due to a shift from anabolism to catabolism. Sugar signaling involves multiple partially redundant regulatory networks that are principally mediated by three multifunctional sensor complexes: hexokinase (HXK1), Snf1-related protein kinases (SnRK1) and target of rapamycin (TOR). SnRK1 is a protein kinase that, under certain low sugar conditions, phosphorylates more than a thousand target proteins. This phosphorylation stimulates catabolism and represses anabolism, thus promoting sugar availability. Conversely, under certain high sugar conditions, SnRK1 becomes inhibited and result in a shift from catabolism to anabolism. This shift may mediate sugar homeostasis.

Fatty acid (FA) synthesis depends on the supply of photosynthetically-derived sugar as a source of carbon skeletons, ATP and reductant. Different factors influence the accumulation of triacylglycerol (TAG) in vegetative tissues. Factors that increase lipid synthesis and accumulation can be referred to as “push,” “pull” and “protect.” Examples of push factors include: WRINKLED1 (WRI1), an APETALA2 (AP2) transcriptional factor that induces the expression of more than 20 genes involved in glycolysis and FA synthesis; and trigalactosyldiacylglycerol 1 (TGD1), a permease-like protein involved in lipid reimport from the endoplasmic reticulum (ER) to the plastid that, when mutated, stimulate FA synthesis and TAG accumulation. Pull factors include various acyltransferases such as, for example, diacylglycerol acyltransferase 1 (DGAT1) which may catalyze the formation of TAG from diacylglycerol and acyl-CoA; and phospholipid:diacylglycerol acyltransferase (PDAT) which catalyzes the formation of TAG by an acyl transfer from the sn-2 position of phospholipids to diacylglycerols (DAG). Protect factors may include for example OLEOSIN1 (OLE1), an amphipathic lipid body-associated protein that influences oil body size and physically protects TAG from hydrolysis by lipases such as, for example, SUGAR-DEPENDENT1 (SDP1), a TAG lipase in Arabidopsis.

There may be a dependency between FA and TAG synthesis with that of sugar availability, through an interrelationship between the regulation and coordination of these processes. Further there may be links between sugar signaling and the posttranslational regulation of lipid metabolism. For example, a putative SnRK1 target site at Ser-197 identified in a nasturtium DGAT1 may be mutated to Ala for increased DGAT activity, and overexpression of the mutant in Arabidopsis seed may result in increased oil accumulation. Sugar may potentiate the oleogenic effects of the WRI1 transcription factor in vegetative tissues. A mechanistic explanation for sugar potentiation of WRI1 may be that SnRK1, encoded by KIN10 and KIN11 in Arabidopsis, phosphorylates two previously unidentified SnRK1 target sites within WRI1 that marks it for proteasomal degradation. According to such a model, under certain low sugar conditions, when SnRK1 is active, WRI1 is degraded and lipid synthesis repressed. Conversely, when sugar is under certain high sugar conditions, SnRK1 is repressed and WRI1 is stabilized and lipid synthesis is stimulated.

In photosynthesizing leaves, sucrose (Suc) is synthesized primarily in mesophyll cells, from where it is transported to phloem in a two-step process. SWEET proteins mediate the passive release of Suc from photosynthetic cells to the apoplast. Sucrose is subsequently loaded from the apoplast into the phloem by an expressed Suc-proton symporter 2 (SUC2). Certain Arabidopsis lines with mutations in SUC2 result in impaired Suc loading into the phloem, or in other words, a decreased sucrose export from source leaves. The decrease in Suc export from source leaves starves nonphotosynthetic sink tissues and may stunt growth of the phenotype. Relative to wild-type Arabidopsis, suc2-4 shows a 20-fold increase in the levels of transient carbohydrate (i.e., the sum of glucose, fructose, Suc and starch). The majority of transient carbohydrate in suc2-4 leaves accumulates as starch, the synthesis of which competes with lipid synthesis for carbon skeletons. Elevated sugar in suc2-4 also results in the induction of chalcone synthase (TT4), a polyketide synthase type III enzyme that mediates the first committed step in flavonoid synthesis that may lead to anthocyanins.

The first committed step in starch synthesis may be ATP: α-glucose-1-phosphate adenylyl transferase, also known as ADP-Glc pyrophosphorylase (ADGase). ADGase catalyzes the formation of ADP-Glc from Glc-1-phosphate and ATP. ADGase is composed of small subunits (e.g., 56 kDa) and large subunits (57 kDa) encoded by ADG1 and ADG2, respectively. In adg1 mutant plants, neither ADG1 nor ADG2 accumulates and the plants are devoid of ADGase activity.

### SUMMARY OF THE INVENTION

It has been surprisingly found that combining certain genetic mutations in plants increases sugar levels in the vegetative tissues of the plants, particularly, in plant leaves. The specific combinations of mutations decrease the transport of sugar out of leaves and decrease the conversion of sugars to starch. It has been found that the increase in sugar accumulation yields increased oil production by stabilizing the oil on-switch, and also by supplying the carbon building blocks needed to make oil in vegetative tissues.

In one embodiment, the plant, or a vegetative plant tissue thereof, comprises two mutations, wherein the two mutations are an adg1 mutation and a suc2 mutation, wherein leaves of the plant have at least about 50-fold more sugar than a counterpart wild type plant.

In one embodiment, the plant, or a vegetative plant tissue thereof, comprising three mutations, wherein the three mutations are an adg1 mutation, a suc2 mutation and a sdp1 mutation, or comprising an adg1 mutation, a suc2 mutation and a tt4 mutation, wherein leaves of the plant have at least about 25% higher triacylglycerol content than a counterpart plant comprising an adg1 mutation and a suc2 mutation.

In one embodiment, the plant, or a vegetative plant tissue thereof, comprising four mutations, wherein the four mutations are an adg1 mutation, a suc2 mutation, an sdp1 mutation, and a tgd1 mutation, or comprising an adg1 mutation, a suc2 mutation, a tt4 mutation and a tgd1 mutation, wherein leaves of the plant have at least about 10 times greater triacylglycerol content than a counterpart wild type plant.

Examples of typical plants used in the invention include plants from the Brassicaceae family or Solanaceae family. Examples of typical plant species include Camelina sativa, Thlaspi arvense, Brassica napus, Arabidopsis thaliana, Nicotiana tobaccum and Nicotiana benthamiana.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1(A) illustrates an example of a stunted phenotype of suc2 partially rescued in adg1suc2. Phenotypes of the shoots of wild type (WT), adg1, suc2 and adg1suc2. Plants were grown on soil for six weeks;

FIG. 1(B) illustrates an example of a stunted phenotype of suc2 partially rescued in adg1suc2. Root growth of 2-week old seedlings of the indicated genotypes grown on the 1/2MS plate (top). The lengths of primary roots of the plants are graphed (bottom). Values are means±SD for each genotype. Levels indicated with different letters above histogram bars are statistically significantly different (Student's t test for all pairs of genotypes, n=10, P<0.05). The experiment was repeated three times, and data from one representative experiment are shown;

FIG. 2(A) illustrates an example of an accumulation of glucose and sucrose in adg1suc2 leaves. Iodine staining showed that starch is not accumulated in adg1suc2 at the end of light period. Leaves were developmentally matched vegetative rosette leaves harvested from equivalent positions from 4-week-old plants at the end of light cycle;

FIG. 2(B) illustrates an example of an accumulation of glucose and sucrose in adg1suc2 leaves. Glucose and sucrose content in the leaves of wild type (WT), adg1, suc2 and adg1suc2 4-week-old soil-grown plants. FW is fresh weight. Values are means±SD for each genotype. Levels indicated with different letters above histogram bars are statistically significantly different (Student's t test, n=5, P<0.05). The experiment was repeated three times, and data from one representative experiment are shown;

FIG. 3(A) illustrates an example of high sugar content in adg1suc2 that activates anthocyanin synthesis and regulates gene expression. Quantification of monomeric anthocyanins in adg1suc2. Asterisks denote statistically significant difference from wild type (WT) (Student's t test, n=5,**, P<0.01). DW is dry weight. The experiment was repeated three times, and data from one representative experiment are shown;

FIG. 3(B) and FIG. 3(C) illustrates an example of high sugar content in adg1suc2 that activates anthocyanin synthesis and regulates gene expression. Gene expression of Chalcone synthase (TT4) and TPS5, respectively, are significantly increased in adg1suc2 mutants;

FIG. 3(D) illustrates an example of high sugar content in adg1suc2 that activates anthocyanin synthesis and regulates gene expression. Gene expression of Glc (Suc) transporters on vacuolar membrane: SUC4, TMT1/2 are not significantly different between WT and adg1suc2. The gene expression values represent means±SD of measurements obtained by quantitative real time PCR (qRT-PCR) employing the primers shown in Table 1. F-box gene expression was used as a control for normalization. Significance was determined by mean crossing point deviation analysis computed by the relative expression software algorithm (REST). Levels indicated with different letters above histogram bars are significantly different (P<0.05). The experiments were repeated three times, and data from one representative experiment are shown;

FIG. 4(A) illustrates TAG accumulation in leaves of adg1suc2. Lipid droplets are abundant in leaves of adg1suc2. Representative confocal fluorescence micrographs of mesophyll tissue from WT and adg1suc2 after 6-weeks of growth on soil after staining with BODIPY 493/503. DIC, differential interference contrast image, Bar=50 μm;

FIG. 4(B) and FIG. 4(C) FIG. 4 illustrates TAG accumulation in leaves of adg1suc2. TAG and total FA, respectively, were quantified from 6-week-old leaves of soil-grown plants as indicated;

FIG. 4(D) illustrates TAG accumulation in leaves of adg1suc2. [1-¹⁴C] Acetate incorporation into fatty acyl products by leaf strips after 30 min of labeling. OWD, the line coexpressing WRI1, DGAT1 and OLE1 is used as a positive control for labeling assay; values in this figure are means±SD for each genotype; levels indicated with different letters above histogram bars are significantly different (Student's, t test, n=5, P<0.05). Each experiment was repeated three times, and data from one representative experiment are shown;

FIG. 5(A) illustrates WRI1 accumulated to higher levels in adg1suc2 than in WT leaves. Gene expression of WRI1 in adg1suc2 is not statistically significantly different from that in WT as determined by mean crossing point deviation analysis computed by the relative expression software algorithm (REST). The experiment was repeated three times, and data from one representative experiment are shown;

FIG. 5(B) illustrates WRI1 accumulated to higher levels in adg1suc2 than in wild type leaves. WRI1 protein accumulates to higher levels in adg1suc2 than in WT. OWD, a transgenic line coexpressing WRI1, DGAT1 and OLE1, is used as a positive control for WRI1 polypeptide expression. Histone H3 is used as a control for protein load. M indicates protein markers;

FIG. 6(A) illustrates an introduction of additional mutations: tt4, sdp1 and tgd1 to further increase TAG and/or total FA content when combined with the adg1suc2. Phenotypes of the shoots of 4-week-old plants of indicated genotypes. Bar=20 mm;

FIG. 6(B) illustrates an introduction of additional mutations: tt4, sdp1 and tgd1 to further increase TAG and/or total FA content when combined with the adg1suc2. TAG was quantified in leaves of WT, double mutant: adg1suc2, triple mutants: adg1suc2tt4, adg1suc2sdp1, and quadruple mutant: adg1suc2tt4tgd1 (left). Total FA was also quantified in corresponding leaves of indicated genotypes (right). Values are means±SD for each genotype. Levels indicated with different letters above histogram bars are significantly different (Student's t test for all pairs of genotypes, n=5, P<0.05); each experiment was repeated three times, and data from one representative experiment are shown;

FIG. 7(A) illustrates co-expression of WRI1, DGAT1 and OLE1 (OWD) in adg1suc2 increases the accumulation of TAG and total FA in adg1suc2. TAG quantification in leaves of 5-week-old WT, double mutant adg1suc2, and the same lines expressing OWD;

FIG. 7(B) illustrates co-expression of WRI1, DGAT1 and OLE1 (OWD) in adg1suc2 increases the accumulation of TAG and total FA in adg1suc2. Total FA was also quantified in leaves from plants described in FIG. 7(A);

FIG. 7(C) illustrates co-expression of WRI1, DGAT1 and OLE1 (OWD) in adg1suc2 increases the accumulation of TAG and total FA in adg1suc2. Compositions of the FA pools of WT and the indicated genotypes from the present studies. Values are means±SD for each genotype. Levels indicated with different letters above histogram bars are statistically significantly different (Student's t test for all pairs of genotypes, n=5, P<0.05). Each experiment was repeated three times and data from one representative experiment are shown;

FIG. 8 (A) illustrates phenotyping the root growth of 2 week old seedlings of the indicated genotypes grown on 1/2MS plate supplemented with 1% sucrose, placed vertically.

FIG. 8 (B) illustrates the length of primary roots of corresponding genotypes. Levels not connected by the same letter in histogram are significantly different (Student's t test, n=5, P<0.05); and

FIG. 9 (A) illustrates transient expression of OWD in tobacco leaves. T-DNA construct designed for constitutive co-expression of three genes: Cys-OLE1, WRI1 and DGAT1 (OWD);

FIG. 9(B) illustrates transient expression of OWD in tobacco leaves. Laser scanning confocal images showed the co-expression of WRI1 and OLE1 in the same epidermis cells of N. benthaminana leaves transiently-transformed with OWD. Bar=50 μm;

FIG. 9(C) illustrates transient expression of OWD in tobacco leaves. TAG levels in N. benthaminana leaves that were transiently-transformed with EV (empty vector), OLE1-GFP+CFP-WRI (OW) or OWD. C is non-infiltrated leaves. ** denotes statistically significant difference from EV (Student's t test, P<0.01);

all arranged according to at least some embodiments described herein.

### DETAILED DESCRIPTION OF THE INVENTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

In one aspect, the invention provides mutant plants, and plant parts thereof, wherein the vegetative parts of the plant have increased fatty acid (FA) content and increased triacylglycerol (TAG) content vis-à-vis wild type (WT) plants of the same species, i.e., wild type counterpart plants. Wild type plants do not comprise the mutations described herein.

As used herein, the term “plant” includes reference to an immature or mature whole plant, including a plant from which seed, grain, or anthers have been removed. Seed or embryo that will produce the plant is also considered to be the plant.

As used herein, the term “plant part” (or “parts thereof”) includes, but is not limited to, protoplasts, leaves, stems, roots, root tips, anthers, pistils, seed, grain, embryo, pollen, ovules, cotyledon, hypocotyl, pod, flower, shoot, tissue, petiole, cells, meristematic cells, and the like.

The plants of the present invention include all terrestrial plants, typically crop plants. Some examples of suitable plants include plants of the Brassicaceae family and Solanaceae family. Some examples of plants of the Brassicaceae family include those of the following genera Camelina, Thlaspi, Brassica and Arabidopsis. Some examples of plant species of the Brassicaceae family include C. sativa, T. arvense, B. napus and A. thaliana. Some examples of plants of the Solanaceae family include those of the genus Nicotiana. Some examples of plants of the Nicotiana genus include the following species N. tobaccum and N. benthamiana.

### Double Mutant Plants

In one embodiment, plants comprising an adg1 mutation and a suc2 mutation are provided. Without wanting to be bound by a mechanism, it is believed that the adg1 mutation disables a small subunit of ADP-glucose pyrophosphorylase, the first step in starch synthesis; and the suc2 mutation disables a sucrose/proton symporter that facilitates sucrose loading from leaves into phloem. Plants with these mutations are referred to herein as adg1suc2 double mutant plants, or as adg1suc2 double mutants.

The adg1suc2 double mutants have an increased sugar content (e.g., glucose and sucrose) in their vegetative tissues (e.g., leaf tissue) by a factor of at least about 40, about 50, about 60, about 70, about 80, about 90, or about 100, relative to their wild type counterparts. For example, the increase in leaf sugar content in adg1suc2 double mutants is at least about 40 to about 200 times that of a wild type counterpart plant leaf.

The adg1suc2 double mutants have an increased total fatty acid (FA) content in their vegetative tissues (e.g., leaf tissue). The increased FA content is at least about 0.5-fold higher, about 1-fold higher, about 1.5-fold higher, about 1.8-fold higher, or about 2.0-fold higher, relative to their wild type counterparts. A 1-fold increase is a 100% increase. For example, the increase in leaf FA content is at least about 50% to about 400% that of a wild type counterpart plant leaf.

The adg1suc2 double mutants have an increased triacylglycerol (TAG) content in their vegetative tissues (e.g., leaf tissue). The increased TAG content is at least about 5-fold higher, about 7-fold higher, about 9-fold higher, about 10-fold higher, about 12-fold higher, about 15-fold higher, about 17-fold higher, or about 20-fold higher, relative to their wild type counterparts. For example, the increase in leaf TAG content is at least about 500% to about 4000% that of a wild type counterpart plant leaf.

### Triple Mutant Plants

In one embodiment, plants comprising three mutations are provided. In particular, in addition to the adg1 and suc2 mutations, these plants comprise a sdp1 mutation. Without wanting to be bound by a mechanism, it is believed that the sdp1 mutation suppresses SDP1 TAG lipase. Plants with these mutations are referred to herein as adg1suc2sdp1 triple mutant plants, or as adg1suc2sdp1 triple mutants.

The adg1suc2sdp1 triple mutants have an increased TAG content in their vegetative tissues (e.g., leaf tissue). The increased TAG content is at least about 25% higher, about 30% higher, about 40% higher, about 50% higher, about 60% higher, about 66% higher, about 70% higher, or about 80% higher, relative to their adg1suc2 double mutant counterparts. For example, the increase in leaf TAG content is about 25% to about 200% that of an adg1suc2 double mutant counterpart plant leaf.

In one embodiment, plants comprising the following three mutations are provided: adg1, suc2 and tt4. Without wanting to be bound by a mechanism, it is believed that the tt4 mutation disables chalcone synthase. Plants with these mutations are referred to herein as adg1suc2tt4 triple mutant plants, or as adg1suc2tt4 triple mutants.

The adg1suc2tt4 triple mutants have an increased TAG content in their vegetative tissues (e.g., leaf tissue). The increased FA and TAG content is at least about 5% higher, about 10% higher, about 15% higher, about 20% higher, about 25% higher, about 30% higher, about 35% higher, or about 40% higher, relative to their adg1suc2 double mutant counterparts. For example, the increase in leaf FA and/or TAG content is about 5% to about 100% that of an adg1suc2 double mutant counterpart plant leaf.

### Quadruple Mutant Plants

In one embodiment, plants comprising four mutations are provided. In addition to the adg1, suc2 and sdp1 mutations, these plants comprise a tgd1 mutation. Without wanting to be bound by a mechanism, it is believed that the tgd1mutation disables an importer of lipids into plastids. Plants with these mutations are referred to herein as adg1suc2sdp1tgd1 quadruple mutant plants, or as adg1suc2sdp1tgd1 quadruple mutants.

The adg1suc2sdp1tgd1 quadruple mutants have an increased TAG content in their vegetative tissues (e.g., leaf tissue). The increased TAG content is at least about 1.5 times higher, about 2 times higher, about 3 times higher, or about 5 times, relative to their adg1suc2sdp1 triple mutant counterparts. For example, the increase in leaf TAG content is at least about 2 times to about 10 times that of an adg1suc2sdp1 triple mutant counterpart plant leaf.

In one embodiment, plants comprising the following four mutations are provided: adg1, suc2, tt4 and tgd1. Plants with these mutations are referred to herein as adg1suc2tt4tgd1 quadruple mutant plants, or as adg1suc2tt4tgd1 quadruple mutants.

The adg1suc2tt4tgd1 quadruple mutants have an increased TAG content in their vegetative tissues (e.g., leaf tissue) vis-à-vis wild type counterpart plants. The increased TAG content is at least about 10 fold higher, about 20 fold higher, about 30 fold higher, or about 50 fold higher, relative to their wild type counterparts. For example, the increase in leaf TAG content is at least about 10 times to about 100 times that of a wild type counterpart plant leaf.

### Methods of Making the Mutant Plants

The double, triple or quadruple mutants can be generated by crossing the corresponding mutant lines. The genotyping primers used to identify homozygous mutants are listed in Table 1.

Additional methods to make the mutant plants include, but are not limited to, expression vectors introduced into plant tissues using a direct gene transfer method, such as microprojectile-mediated delivery, DNA injection, electroporation, and the like, thereby yielding the mutant plants. More typically, expression vectors are introduced into plant tissues by using either microprojectile-mediated delivery with a biolistic device or by using Agrobacterium-mediated transformation. Any DNA sequences, whether from a different species or from the same species, which are introduced into the genome using transformation or various breeding methods are referred to herein collectively as “transgenes.”

Numerous methods for plant transformation have been developed, including biological and physical plant transformation protocols. See, for example, Miki et al., “Procedures for Introducing Foreign DNA into Plants,” in Methods in Plant Molecular Biology and Biotechnology, Glick and Thompson Eds., CRC Press, Inc., Boca Raton, pp. 67-88 (1993), and Armstrong, “The First Decade of Maize Transformation: A Review and Future Perspective,” Maydica, 44:101-109 (1999). In addition, expression vectors and in vitro culture methods for plant cell or tissue transformation and regeneration of plants are available. See, for example, Gruber, et al., “Vectors for Plant Transformation,” in Methods in Plant Molecular Biology and Biotechnology, Glick and Thompson Eds., CRC Press, Inc., Boca Raton, pp. 89-119 (1993).

A genetic trait which has been engineered into the genome of a particular plant may then be moved into the genome of another variety using traditional breeding techniques that are well known in the plant breeding arts. For example, a backcrossing approach is commonly used to move a transgene from a transformed plant variety into an already developed plant variety, and the resulting backcross conversion plant would then comprise the transgene(s). Backcrossing is a process in which a breeder crosses progeny back to one of the parental genotypes one or more times.

Various genetic elements can be introduced into the plant genome using transformation. These elements include, but are not limited to, genes, coding sequences, inducible, constitutive and tissue specific promoters, enhancing sequences, and signal and targeting sequences.

Plant transformation involves the construction of an expression vector which function in plant cells. Such a vector comprises DNA comprising a gene under control of, or operatively linked to, a regulatory element (for example, a promoter). The expression vector may contain one or more such operably linked gene/regulatory element combinations. The vector(s) may be in the form of a plasmid and can be used alone or in combination with other plasmids to incorporate transgenes into the genetic material of plants.

Expression vectors include at least one genetic marker operably linked to a regulatory element (for example, a promoter) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin. Fraley, et al., Proc. Natl. Acad. Sci. USA, 80:4803 (1983). Another commonly used selectable marker gene is the hygromycin phosphotransferase gene which confers resistance to the antibiotic hygromycin. Vanden Elzen, et al., Plant Mol. Biol., 5:299 (1985).

Additional selectable marker genes of bacterial origin that confer resistance to antibiotics include gentamycin acetyl transferase, streptomycin phosphotransferase and aminoglycoside-3′-adenyl transferase, the bleomycin resistance determinant (Hayford, et al., Plant Physiol., 86:1216 (1988); Jones, et al., Mol. Gen. Genet., 210:86 (1987); Svab, et al., Plant Mol. Biol., 14:197 (1990); Hille, et al., Plant Mol. Biol., 7:171 (1986)). Other selectable marker genes confer resistance to herbicides such as glyphosate, glufosinate, or bromoxynil (Comai, et al., Nature, 317:741-744 (1985); Gordon-Kamm, et al., Plant Cell, 2:603-618 (1990); Stalker, et al., Science, 242:419-423 (1988)).

Selectable marker genes for plant transformation not of bacterial origin include, for example, mouse dihydrofolate reductase, plant 5-enolpyruvylshikimate-3-phosphate synthase, and plant acetolactate synthase (Eichholtz, et al., Somatic Cell Mol. Genet., 13:67 (1987); Shah, et al., Science, 233:478 (1986); Charest, et al., Plant Cell Rep., 8:643 (1990)).

Another class of marker genes for plant transformation requires screening of presumptively transformed plant cells, rather than direct genetic selection of transformed cells, for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include β-glucuronidase (GUS), β-galactosidase, luciferase, and chloramphenicol acetyltransferase (Jefferson, R. A., Plant Mol. Biol. Rep., 5:387 (1987); Teeri, et al., EMBO J., 8:343 (1989); Koncz, et al., Proc. Natl. Acad. Sci. USA, 84:131 (1987); DeBlock, et al., EMBO J., 3:1681 (1984)).

In vivo methods for visualizing GUS activity that do not require destruction of plant tissue are available (Molecular Probes, Publication 2908, IMAGENE GREEN, pp. 1-4 (1993); Naleway, et al., J. Cell Biol., 115:151a (1991)). Also, a gene encoding Green Fluorescent Protein (GFP) can be utilized as a marker for gene expression in prokaryotic and eukaryotic cells (Chalfie, et al., Science, 263:802 (1994)). GFP and mutants of GFP may be used as screenable markers.

Genes included in expression vectors must be driven by a nucleotide sequence comprising a regulatory element (for example, a promoter). Several types of promoters are well known in the transformation arts as are other regulatory elements that can be used alone or in combination with promoters. As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters that initiate transcription only in a certain tissue are referred to as “tissue-specific.” A “cell-type” specific promoter primarily drives expression in certain cell types in one or more organs, for example, vascular cells in roots or leaves. An “inducible” promoter is a promoter which is under environmental control. Examples of environmental conditions that may affect transcription by inducible promoters include anaerobic conditions or the presence of light. Tissue-specific, tissue-preferred, cell-type specific, and inducible promoters constitute the class of “non-constitutive” promoters. A “constitutive” promoter is a promoter that is active under most environmental conditions.

A. Inducible Promoters—An inducible promoter is operably linked to a gene for expression in a plant. Optionally, the inducible promoter is operably linked to a nucleotide sequence encoding a signal sequence which is operably linked to a gene for expression in a plant. With an inducible promoter the rate of transcription increases in response to an inducing agent. Any inducible promoter can be used in the instant invention. See, Ward, et al., Plant Mol. Biol., 22:361-366 (1993). Exemplary inducible promoters include, but are not limited to, that from the ACEI system which responds to copper (Mett, et al., Proc. Natl. Acad. Sci. USA, 90:4567-4571 (1993)); In2 gene from maize which responds to benzenesulfonamide herbicide safeners (Hershey, et al., Mol. Gen Genetics, 227:229-237 (1991); Gatz, et al., Mol. Gen. Genetics, 243:32-38 (1994)); or Tet repressor from Tn10 (Gatz, et al., Mol. Gen. Genetics, 227:229-237 (1991)). A typical inducible promoter is a promoter that responds to an inducing agent to which plants do not normally respond. An exemplary inducible promoter is the inducible promoter from a steroid hormone gene, glucocorticoid response elements, the transcriptional activity of which is induced by a glucocorticoid hormone (Schena, et al., Proc. Natl. Acad. Sci. USA, 88:10421-10425 (1991)).
B. Constitutive Promoters—A constitutive promoter is operably linked to a gene for expression in a plant or the constitutive promoter is operably linked to a nucleotide sequence encoding a signal sequence which is operably linked to a gene for expression in a plant. Many different constitutive promoters can be utilized in the instant invention. Exemplary constitutive promoters include, but are not limited to, the promoters from plant viruses such as the 35S promoter from CaMV (Odell, et al., Nature, 313:810-812 (1985)) and the promoters from such genes as rice actin (McElroy, et al., Plant Cell, 2: 163-171 (1990)); ubiquitin (Christensen, et al., Plant Mol. Biol., 12:619-632 (1989); Christensen, et al., Plant Mol. Biol., 18:675-689 (1992)); pEMU (Last, et al., Theor. Appl. Genet., 81:581-588 (1991)); MAS (Velten, et al., EMBO J., 3:2723-2730 (1984)); and maize H3 histone (Lepetit, et al., Mol. Gen. Genetics, 231:276-285 (1992); Atanassova, et al., Plant Journal, 2 (3): 291-300 (1992)). The ALS promoter, an Xbal/Ncol fragment 5′ to the Brassica napus ALS3 structural gene (or a nucleotide sequence similarity to said Xbal/Ncol fragment), represents a particularly useful constitutive promoter. See PCT Application WO 96/30530.
C. Tissue-Specific or Tissue-Preferred Promoters—A tissue-specific promoter is operably linked to a gene for expression in a plant. Optionally, the tissue-specific promoter is operably linked to a nucleotide sequence encoding a signal sequence which is operably linked to a gene for expression in a plant. Plants transformed with a gene of interest operably linked to a tissue-specific promoter produce the protein product of the transgene exclusively, or preferentially, in a specific tissue. Any tissue-specific or tissue-preferred promoter can be utilized in the instant invention. Exemplary tissue-specific or tissue-preferred promoters include, but are not limited to, a root-preferred promoter such as that from the phaseolin gene (Murai, et al., Science, 23:476-482 (1983); Sengupta-Gopalan, et al., Proc. Natl. Acad. Sci. USA, 82:3320-3324 (1985)); a leaf-specific and light-induced promoter such as that from cab or rubisco (Simpson, et al., EMBO J., 4(11):2723-2729 (1985); Timko, et al., Nature, 318:579-582 (1985)); an anther-specific promoter such as that from LAT52 (Twell, et al., Mol. Gen. Genetics, 217:240-245 (1989)); a pollen-specific promoter such as that from Zm13 (Guerrero, et al., Mol. Gen. Genetics, 244:161-168 (1993)); or a microspore-preferred promoter such as that from apg (Twell, et al., Sex. Plant Reprod., 6:217-224 (1993)).

Transport of a protein produced by transgenes to a subcellular compartment, such as the chloroplast, vacuole, peroxisome, glyoxysome, cell wall, or mitochondrion, or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine during protein synthesis and processing where the encoded protein is ultimately compartmentalized. The presence of a signal sequence directs a polypeptide to either an intracellular organelle or subcellular compartment or for secretion to the apoplast. Many signal sequences are known in the art. See, for example, Becker, et al., Plant Mol. Biol., 20:49 (1992); Knox, C., et al., Plant Mol. Biol., 9:3-17 (1987); Lerner, et al., Plant Physiol., 91:124-129 (1989); Frontes, et al., Plant Cell, 3:483-496 (1991); Matsuoka, et al., Proc. Natl. Acad. Sci., 88:834 (1991); Gould, et al., J. Cell. Biol., 108:1657 (1989); Creissen, et al., Plant 1, 2:129 (1991); Kalderon, et al., Cell, 39:499-509 (1984); Steifel, et al., Plant Cell, 2:785-793 (1990).

Partial Rescue of suc2 by Introducing adg1

The dwarf phenotype of suc2 may relate to an inability to export sucrose to developing shoots and roots which may restricts carbon available for growth and development. Combining adg1 with suc2 may partially mitigate the dwarf phenotype with respect to shoot size and primary root elongation and may suggest that more sucrose can be available to shoots and roots in the adg1suc2 double mutant. This may be supported by the observation that adg1suc2 and suc2 seeds that germinate on 1/2MS medium supplemented with sucrose may show equivalent primary root elongation. The partial rescue of suc2 by introducing adg1 may be related to inactivation of the competing starch biosynthesis resulted in a higher intracellular sucrose concentration. This may increase the sucrose gradient across the membranes of phloem companion cells in adg1suc2 relative to that of suc2, thereby driving passive sugar efflux. It may also be possible that certain levels of sugar could activate, or induce the accumulation of other phloem loading sucrose transporters to facilitate increased sucrose transport to shoots and roots.

Sugar Accumulation in adg1suc2 Leaves

Leaves typically may accumulate photosynthetically fixed sugars less than 10 nmole per mg fresh weight (corresponding to approximately that of WT). Photosynthetically fixed sugars in leaves may be converted to starch, a short-term storage carbohydrate in leaves, or they may be loaded into phloem and transported to sink tissues. Therefore, photosynthetically fixed sugars may not accumulate in the leaves. Environmental conditions such as light greater than 500 micro einsten per square meter or low temperatures less than 15° C. can favor sucrose synthesis and accumulation. For example, low temperature may decrease carbon utilization which may allow Suc concentrations to rise. In plants, sucrose may be found naturally in specialized storage organs such as the stems of sugarcane. Specialized organs may have efficient sugar transporters and sugar metabolizing enzymes such as sucrose synthases and invertases, that may facilitate phloem unloading and sugar compartmentalization into the vacuoles of storage parenchyma.

It has been found with the present method that an 80-fold increase of sugars may be achieved in mature, 5-week, Arabidopsis leaves by decreasing sucrose export and blocking starch synthesis simultaneously in the adg1suc2 double mutant. With the present method, elevated levels of Suc may accumulate in the cytosol of adg1suc2. This may be supported by adg1suc2 and wild type having similar levels of expression of TMT1/2, encoding the proton-coupled antiporters capable of high-capacity loading of glucose and sucrose into vacuoles, and similar levels of SUC4, a proton-coupled symporter that exports sucrose from vacuole.

### Sucrose Favors TAG Biosynthesis

Sucrose may have effects on various aspects of plant metabolism. For example, Suc may induce fructan (i.e., polymers of Fru) synthesis in grasses. Sucrose also may act as a signaling molecule that initiates/activates starch synthesis by inducing gene expression of AGPase large subunits, starch synthase (GBSS1) and 3-amylase and by post-translationally activating the AGPase enzyme by redox modification. Suc may downregulate photosynthetic CO₂ fixation. Further an induction of genes related to anthocyanin and flavonoid biosynthesis may occur upon sucrose feeding.

Consistent with exogenous Suc feeding, the strongly elevated levels of intracellular Suc in the present suc2adg1 resulted in dramatic increases in chalcone synthase mRNA and significant accumulation of anthocyanin. The TAG content of 4-week-old Arabidopsis roots cultured on agar supplemented with 3% Suc in a previous study in the prior art was higher than that of roots grown on agar without supplementation because sugar provides carbon skeleton, ATP and reductant for TAG synthesis. In the present study it has been found that the adg1suc2 mutant which accumulates more Suc, accumulates 10-fold more TAG than wild type plants.

In the prior art it was found that glucose supplementation may contribute to TAG accumulation in seedlings ectopically overexpressing WRI1. WRI1 overexpression studies in the prior art may have shown that FA synthesis is potentiated by exogenous sucrose treatment or by increasing intracellular Suc resulting from downregulation of starch synthesis by APGRNAi. These studies may suggest that Suc may be a signaling molecule in regulating the expression of WRI1 target genes.

In the present study, it has been observed that an increase in the level of WRI1 polypeptide in adg1suc2 relative to wild type, which may result from suppression of post-transcriptional KIN10-dependent phosphorylation that predisposes WRI1 to degradation. WRI1 expression, did not differ between adg1suc2 and wild type. The data from the present study, that elevated sugar may correlate with increased accumulation of WRI1, may be consistent with a model in the prior art of WRI1 stabilization upon sucrose-mediated inhibition of KIN10. Therefore WRI1 may be stabilized as a result of a dual sugar activity in providing carbon skeletons for FA synthesis and inhibiting KIN10 activity.

In the storage tissues of some varieties of sugarcane, previous studies in the prior art may indicate that sugar levels can approach 650 mM without stimulating TAG accumulation in stems.

It will be understood that any compound, material or substance which is expressly or implicitly disclosed in the specification and/or recited in a claim as belonging to a group or structurally, compositionally and/or functionally related compounds, materials or substances, includes individual representatives of the group and all combinations thereof.

### Examples

### Materials and Methods

### Plant Materials and Growth Conditions

Seeds of Arabidopsis thaliana ecotype Col-0 (Wild type) and tt4-3 were obtained from Arabidopsis Biological Resource Center (ABRC). The suc2-4 mutant was provided by University of North Texas. In addition, the following mutant lines were used in the present study: suc2-4, adg1-1, sdp1-5 and tgd1. Homozygous double, triple or quadruple mutants were generated by crossing the corresponding mutant lines. The genotyping primers used to identify homozygous mutants are listed in Table 1.

TABLE 1

| Gene | Primer pair sequences (5′-3′) | Purpose |
|---|---|---|
| SUC2 | gtttttcggagaaatcttcgg (SEQ ID NO: 1) and | Genotyping for |
| | caaatgctggaatgtttccac (SEQ ID NO: 2) | suc2 |
| SDP1 | aagcattgaatctggtggttg (SEQ ID NO: 3) and | Genotyping |
| | ttggtgtggttaggactttgg (SEQ ID NO: 4) | |
| TGD1 | atgatgcagacttgttgtatcca (SEQ ID NO: 5) and | Genotyping |
| | cacagttctt caaagaatctcc (SEQ ID NO: 6) | |
| WRI1 | ggggacaagtttgtacaaaaaagcaggcttcatgaagaagcgcttaaccacttc | Gene cloning |
| | (SEQ ID NO: 7) and | |
| | ggggaccactttgtacaagaaagctgggtcttattcagaaccaacgaacaagcc | |
| | (SEQ ID NO: 8) | |
| DGAT1 | ggggacaagtttgtacaaaaaagcaggcttcatggcgattttggattctgctg | Gene cloning |
| | (SEQ ID NO: 9) and | |
| | ggggaccactttgtacaagaaagctgggtctgacatcgatccttttcggttcatc | |
| | (SEQ ID NO: 10) | |
| OLE1 | ggggacaagtttgtacaaaaaagcaggcttcatggcggatacagctagagg | Gene cloning |
| | (SEQ ID NO: 11) and | |
| | ggggaccactttgtacaagaaagctgggtcagtagtgtgctggccacca | |
| | (SEQ ID NO: 12) | |
| TT4 | ggaagtcagctaaggatggtg (SEQ ID NO: 13) and | qRT-PCR |
| | gtccgaaaccaaacaagacac (SEQ ID NO: 14) | |
| TPS5 | aagagcttatggaacacctcg (SEQ ID NO: 15) and | qRT-PCR |
| | agacctttgttcacaccctg (SEQ ID NO: 16) | |
| WRI1 | tcggaagagtgtttgggaac (SEQ ID NO: 17) and | qRT-PCR |
| | caatcgcagccatgtcatatg (SEQ ID NO: 18) | |
| DGAT1 | ccgacgcaatcttcaaacag (SEQ ID NO: 19) and | qRT-PCR |
| | ccgacgcaatcttcaaacag (SEQ ID NO: 20) | |
| PDAT1 | tgttgcagggcttttctctg (SEQ ID NO: 21) and | qRT-PCR |
| | tgttgagtcccatgtgcg (SEQ ID NO: 22) | |
| TMT1 | atgacgcagagatggaacttag (SEQ ID NO: 23) and | qRT-PCR |
| | ggtgtcggcattcaaatactg (SEQ ID NO: 24) | |
| TMT2 | agaactccgttgatgcctg (SEQ ID NO: 25) and | qRT-PCR |
| | acaattggtcctgctatggtac (SEQ ID NO: 26) | |
| SUC4 | cgtcccatatgcgttgatttc (SEQ ID NO: 27) and | qRT-PCR |
| | tcccacctccaaacagttg (SEQ ID NO: 28) | |
| BCCP2 | aacagcaaaaccaacatccg (SEQ ID NO: 29) and | qRT-PCR |
| | cccttctgtaccttatctccaac (SEQ ID NO: 30) | |
| KAS1 | cacaattaacagcacctccaag (SEQ ID NO: 31) and | qRT-PCR |
| | tgggataaagcaagagatggg (SEQ ID NO: 32) | |
| PKP β1 | ctccatacctaacttgcactcc (SEQ ID NO: 33) and | qRT-PCR |
| | ctctgcaccaagatcacctc (SEQ ID NO: 34) | |

For overexpression of OLE1, DGAT1 and WRI1 in adg1-1suc2-4, adg1-1suc2-4 was crossed with an OWD/WT (OLE1, DGAT1 and WRI1 overexpression line in the wild type background). Plants and fresh leaf tissue derived from such plants for the present study were grown with a light/dark cycle of a light period of 16 hours followed by a darkness period of 8 hours, with a day(light)/night (dark) temperature of 23°/19° C., with an applied light that was a photosynthetic photon flux density of 250 μmol m-2 s-1 and with a relative humidity for full light/dark cycling was 75% relative humidity (RH).

RNA Isolation and Quantitative Real Time PCR (qRT-PCR)

To quantify gene expression of chalcone synthase (TT4), TPS5, SUC4, TMT1/2, WRI1, DGAT1 and PDA Ti, total RNAs were isolated from WT and adg1suc2 and treated with DNaseI. cDNA was prepared using SuperScript III First-Strand Synthesis SuperMix (available commercially from Invitrogen). qRT-PCR was performed with the CFX96 qPCR Detection System (commercially available from Bio-Rad) and gene-specific primers for TT4, TPS5, WRI1, DGAT1, PDAT1, F-box (At5g1571) and Expressed1 (AT4G33380) among which F-box and Expressed1 were used as reference genes (see Table 1). Statistical analysis of qRT-PCR data was carried out with REST2009 (Pfaffl et al., 2002).

### Leaf Iodine Staining

For leaf starch iodine staining, fresh leaves sampled from WT, suc2-4 and adg1-1suc2-4 were decolored by incubating with the absolute ethanol at 70° C. for 5 min and before staining with diluted Lugol's solution.

### Leaf Total Anthocyanin, Sucrose and Glucose Quantification

Total leaf anthocyanin levels were quantified by the methods described by Fuleki and Francis (Fuleki and Francis, 1968). For quantification of sucrose and glucose of leaves, assays were performed according to the manufacturers' instructions of Sucrose Assay kit and Glucose (GO) Assay Kit (available commercially from Sigma, St Louis Mo.).

### TAG and Total FA Quantification

Total lipids (TAG and polar lipids) were isolated from 100 mg of fresh leaf tissue prepared as described above in Plant materials and growth conditions section with 700p of Methanol:Chloroform:Formic acid (2:1:0.1, v/v/v) and mixed by vortexing for 30 min. After adding 1 M KCl, 0.2 M H₃PO₄, samples were vortexed and clarified by centrifugation at 2,000×g for 10 min; total lipids were collected as the lower phase. For TAG quantification, 60 μl of total lipids were fractionated by TLC using hexane:diethyl ether:acetic acid (70:30:1, v/v/v) as a mobile phase on Silica Gel 60 plates (commercially available from Merck) and visualized after spraying with 0.05% primuline (in 80% acetone). TAG fractions were identified under UV light, isolated from the plate and transmethylated to FA methyl esters (FAMEs). They were transmethylated by adding 1 ml of boron trichloride-methanol and incubation at 80-85° C. for 40 min. For total FA quantification, 10 μl of total lipids were transmethylated with boron trichloride-methanol. For both assays, 5 μg of C17:0 was added as an internal standard prior to transmethylation. FAMEs were extracted into hexane, and dried under a stream of dry nitrogen before dissolving in 100l hexane. FAMEs were then analyzed using an Agilent Technologies 7890A GC System equipped with a 5975C mass selective detector and a 60 m×0.25 mm ID DB23 column (commercially available from Supelco, Bellefonte, Pa.).

### Antibody and Immunoblotting

Anti-WRI1 and anti-Histone H3 antibodies were used in the present study. Anti-WRI1 polyclonal antibodies were produced using an immunization of a synthetic peptide (DFMFDDGKHECLNLENLDC) (SEQ ID NO: 35) corresponding to residues 299-317 of the WRI1 amino acid sequence (designed and produced by Thermo Scientific Pierce, Thermo Fisher, Waltham Mass.). Histone H3 polyclonal antibodies were purchased from Agrisera. 50 mg of mature leaves of 4-week-old wild type and adg1suc2 were ground under liquid nitrogen and then mixed with 200 μL of preheated protein extraction buffer (8 M urea, 2% SDS, 0.1 M DTT, 20% glycerol, 0.1 M Tris-HCl (pH 6.8), 0.004% bromophenol blue). After clarification by centrifugation at 16,000×g, 30 μl of supernatant was resolved by SDS-PAGE (Sodium Dodecyl Sulphate-Polyacrylamide Gel Electrophoresis) before they were transferred to PVDF (polyvinylidene difluoride) membrane for immunoblot analysis. Immunoblots of target proteins were visualized using alkaline phosphatase-conjugated secondary antibodies and the substrates 5-bromo-4-chloro-3-indolylphosphate/nitro-blue tetrazolium (BCIP/NBT) (available commercially from Bio-Rad, Hercules, Calif.).

In Vivo [1-¹⁴C] Acetate Labeling

[1-¹⁴C] acetate labeling was performed according to the method of Koo et al. (Koo et al., 2004). In brief, half leaves were incubated in 25 mM Na-Morpholinoethanesulfonic acid (MES, pH 5.7) buffer containing 0.01% w/v Tween-20 as a wetting agent under illumination (180 μmol photos m⁻² sec¹) at 25° C. Labeling was initiated by adding 10 μCi of sodium [1-¹⁴C] acetate solution (58 mCi/mmol, commercially available from American Radiolabeled Chemicals). Labeling was terminated by removing the medium and then washing the leaf material three times with DI water. Total lipids were extracted and separated as described above. Radioactivity associated with total lipids was determined by liquid scintillation counting.

### Lipid Staining

Mature leaves from 6-week-old soil grown wild type and adg1-1suc2-4 were cut into approximately 8×2 mm pieces. For BODIPY staining, fresh tissue was incubated with a solution containing 100 μg/mL BODIPY 493/503 (excitation/emission wavelengths) (commercially available from Invitrogen) in 0.1% Triton X-100 (by dilution from a 10 mg/mL DMSO stock solution). Vacuum was applied for 10 min, before washing twice with PBS to remove excess stain. The BODIPY-stained lipid droplets were imaged using a Leica SP5 confocal laser scanning microscope (commercially available from Leica, Buffalo Grove, Ill.) with an excitation wavelength set at 488 nm. Lipid droplets were visualized at 63× magnification, with z gain of 824 and 1200 for BODIPY stain and chlorophyll, respectively.

Introducing adg1 into the suc2 Mutant Background Partially Rescues its Growth Retardation

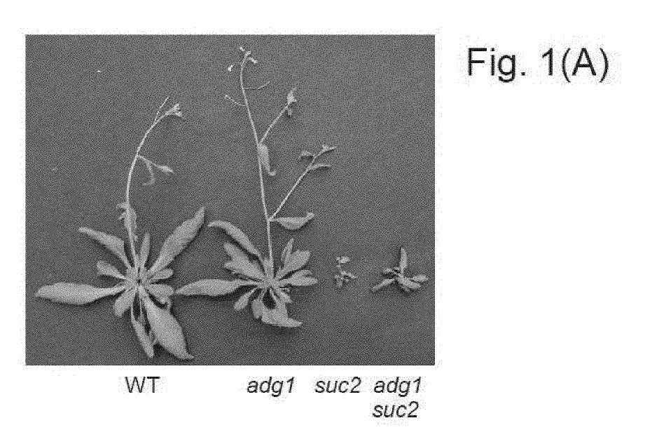

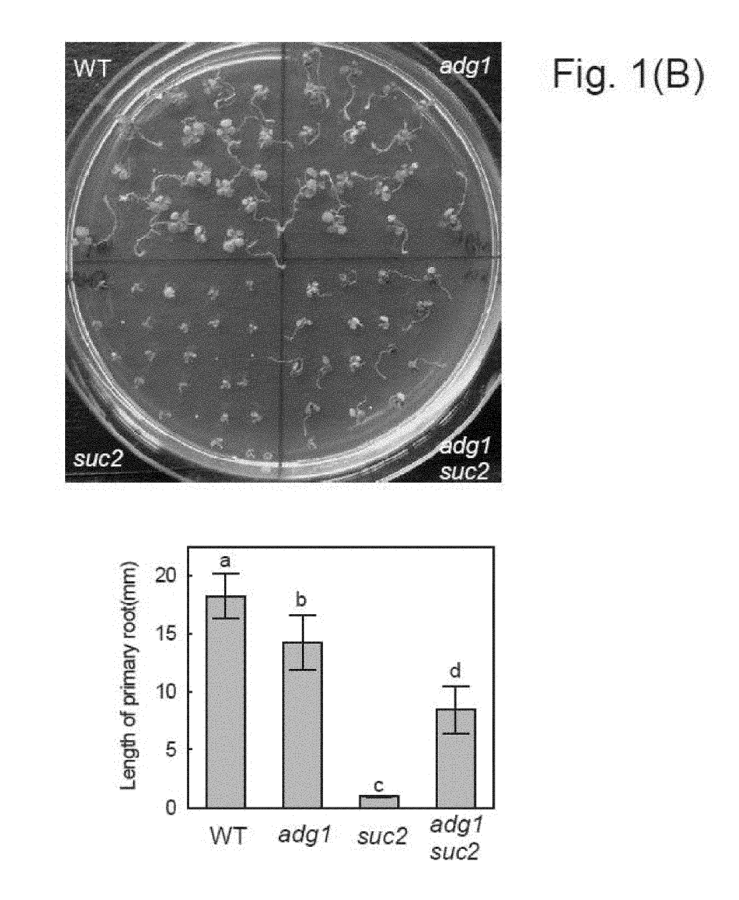

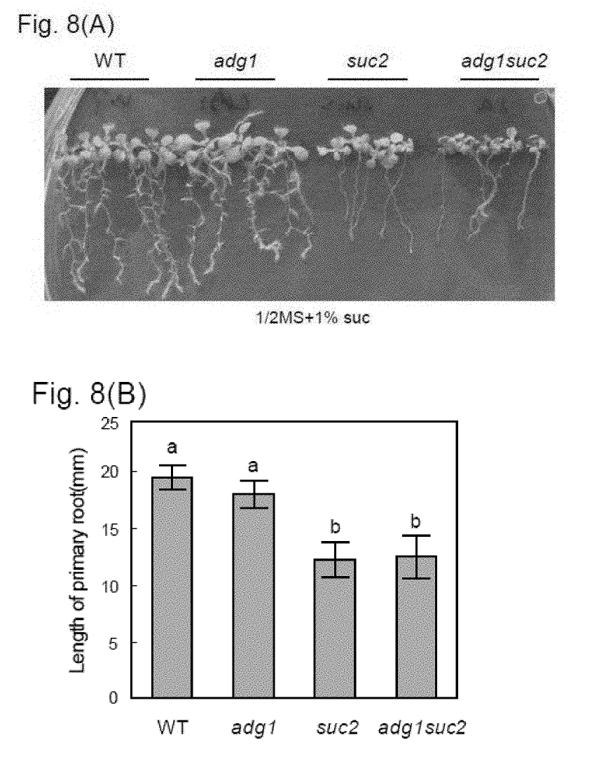

The suc2-4 mutant (Salk_038124, henceforth referred as suc2), a T-DNA insertion mutant line was disrupted in the second intron of AtSUC2 that resulted in a stunted growth phenotype relative to wild type (WT) (FIG. 1A). It was crossed with a starchless mutant adg1-1, which showed similar growth to that of wild type, to generate the adg1-1suc2-4 double mutant (referred to herein as adg1suc2). Combining adg1 with suc2 partially reversed the growth retardation phenotype of the suc2 mutant such that leaves of the double mutant expanded in a more normal manner (FIG. 1A). During seed germination, adg1suc2 mutants also demonstrated more robust primary root elongation than suc2. Primary roots of adg1suc2 were 8.4-fold longer than those of suc2 two weeks after germination and growth on one-half strength Murashige and Skoog (1/2MS) medium without sucrose (FIG. 1B). However, when cultured on 1/2MS media supplemented with 1% sucrose, there was little difference in primary root elongation between adg1suc2 and suc2 (FIG. 8).

Sugar Accumulation in adg1suc2 Mutant

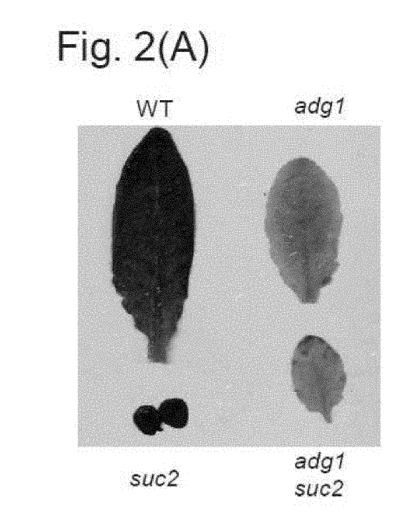

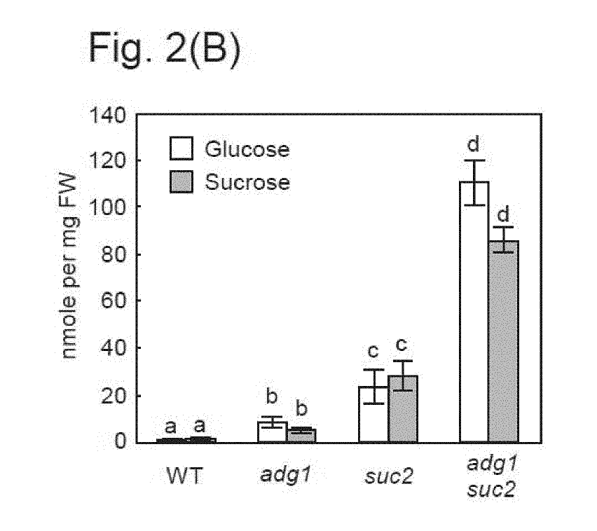

In a phenotype of the adg1 mutant, there was a lack of starch in leaves at the end of a single light period as visualized by iodine staining (FIG. 2A). Similarly, the adg1suc2 double mutant failed to accumulate starch at the end of one light period of a light/dark cycle (i.e., one day) (FIG. 2A). The combined content of glucose and sucrose in the suc2 plant was 21-fold that of wild type, whereas adg1suc2 was 3.8-fold higher than suc2 and 80-fold higher than wild type (FIG. 2B). Leaves of adg1suc2 plants displayed a visibly reddish hue consistent with the accumulation of anthocyanins. Analysis of these leaves revealed that they accumulated 218-fold more anthocyanin than that of wild type plants, corresponding to approximately 1.5% of dry weight.

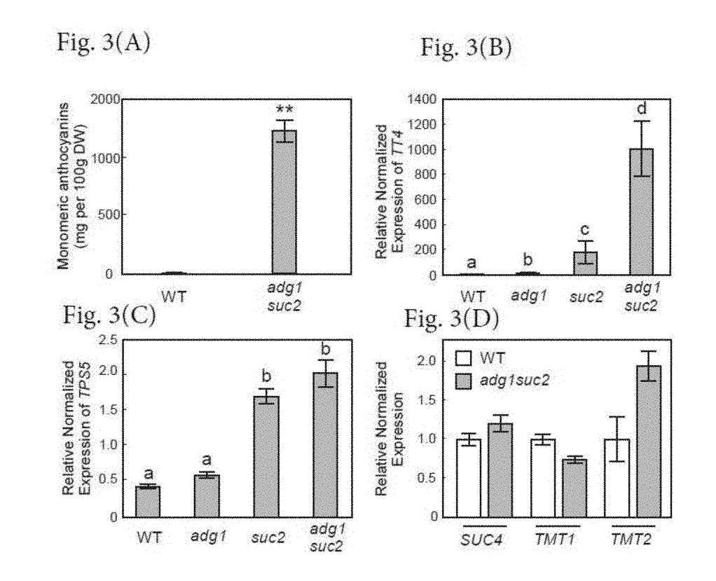

The expression levels of two genes TT4 and TPS5 were also investigated. The expression levels of these genes had previously been demonstrated to be induced upon incubation with sucrose, i.e., chalcone synthase (TT4), an enzyme involved in the biosynthesis of flavonoids and TREHALOSE-6-PHOSPHATASE SYNTHASE 5 (TPS5), an enzyme putatively involved in trehalose metabolism. Though TPS5 may be a class II TPS, it is not catalytically active in producing T6P. To test whether TT4 and TPS5 were also induced by endogenous sucrose accumulation, qRT-PCR was used to quantify their expression in leaves of wild type and adg1suc2 double mutant. The expression of TT4 in adg1suc2 was approximately 6-fold higher than in suc2 and approximately 1000-fold higher than that of wild type (FIG. 3B). Expression of TPS5 was also upregulated in adg1suc2 and suc2 mutants although to a lower degree than that of TT4 (FIG. 3C).

The qRT-PCR data of the present study showed that gene expression of TMT1/2 and SUC4 in adg1suc2 does not differ significantly from that of wild type (FIG. 3D), showing that their transcription was not regulated by sucrose availability.

TAG Accumulation in Leaves of the adg1suc2 Double Mutant

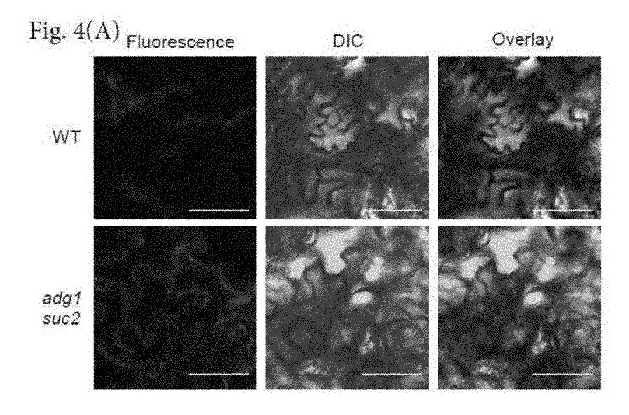

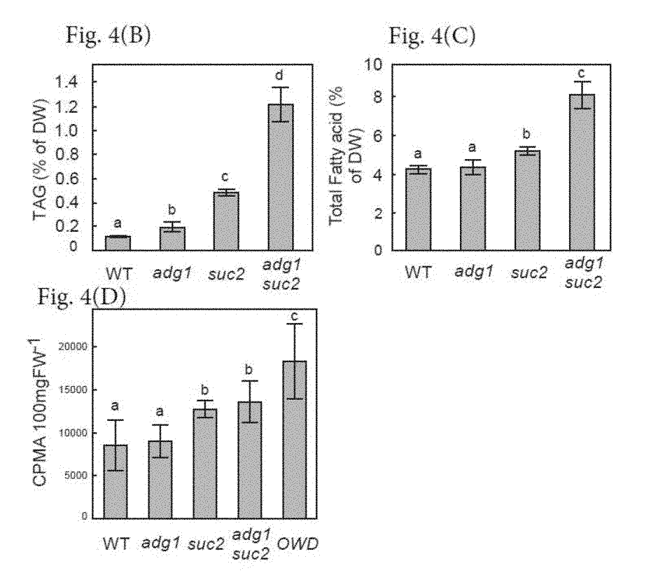

Accumulation of glucose and sucrose in adg1suc2 leaves may assist in a study of TAG biosynthesis in leaves tissues with elevated sugar content. Compared with wild type, adg1suc2 showed more lipid droplets in leaves when visualized with a non-polar lipid-selective fluorescent dye, BODIPY® 493/503 (available commercially from Invitrogen) (FIG. 4A). TAG quantification of mature leaves of 5-week-old plant showed that adg1suc2 accumulated 1.2% TAG (of DW) which was approximately 12-fold higher than that of wild type (FIG. 4B). Correspondingly, leaf total FA content in adg1suc2 was 1.8-fold higher than wild type and reached 8.3% (of DW) (FIG. 4C). A 30 minute [1-¹⁴C] acetate labeling experiment was used to test if endogenous sugar greater than 10 nmole per mg fresh weight (corresponding to approximately 10-fold higher than that of WT) increases de novo FA synthesis, in a similar manner to that observed upon the coexpression of OLEOSIN1, WRI1 and DGA Tin wild type, a combination of genes previously shown to increase TAG accumulation used as a positive control. The relative rate of FA synthesis in leaves of adg1suc2 was 58% higher than that of wild type but was not significantly different from that of suc2 (FIG. 4D)

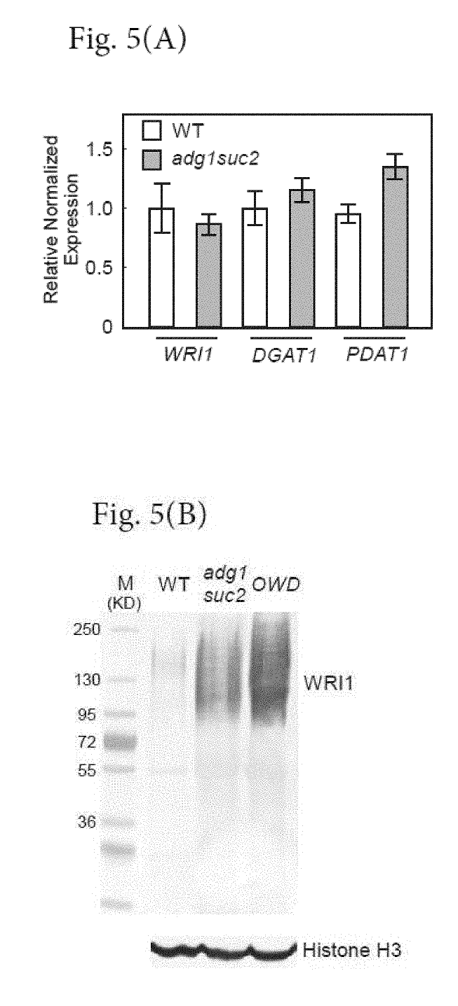

To test whether high endogenous sucrose (e.g., greater than 10 nmole per mg fresh weight (corresponding to approximately 10-fold higher than that of WT) also affects WRI1 in adg1suc2, both gene expression and protein level of WRI1 were quantified by qRT-PCR and immunoblotting, respectively. The results showed gene expression level was not different between WT and adg1suc2 (FIG. 5A), however, while the WRI1 protein was hard to detect in wild type leaves, its levels were higher in leaves of adg1suc2 (FIG. 5B). For comparison, three oleogenic genes, Cys-OLE1, WRI1, and DGAT1, were assembled into a single construct, OWD. Equal loading was demonstrated by performing duplicate western analysis using the constitutive histone H3; however a presumptive Rubisco band appears somewhat stronger in the double mutant and OWD. To visualize expression, cyan fluorescent protein (CFP) was fused at the N-terminus of WRI1 while GFP was fused at C-terminus of Cys-OLE1, generating a cys-OLE-GFP, CFP-WRI and DGAT1 (OWD) construct (SFIG. 2A). In this experiment, the ectopic expression of WRI1 was used as a positive control for the WRI1 western blot. The pattern of expression of WRI1 polypeptide in adg1suc2 was similar to that of OWD expression in wild type. Together these data may indicate that the WRI1 polypeptide was stabilized in adg1suc2, and that increased abundance of WRI1 may be associated with increased fatty acid synthesis and increased TAG accumulation.

Stacking Additional Gene Mutations with adg1suc2 Results in Further Increases TAG and Total FA Accumulation

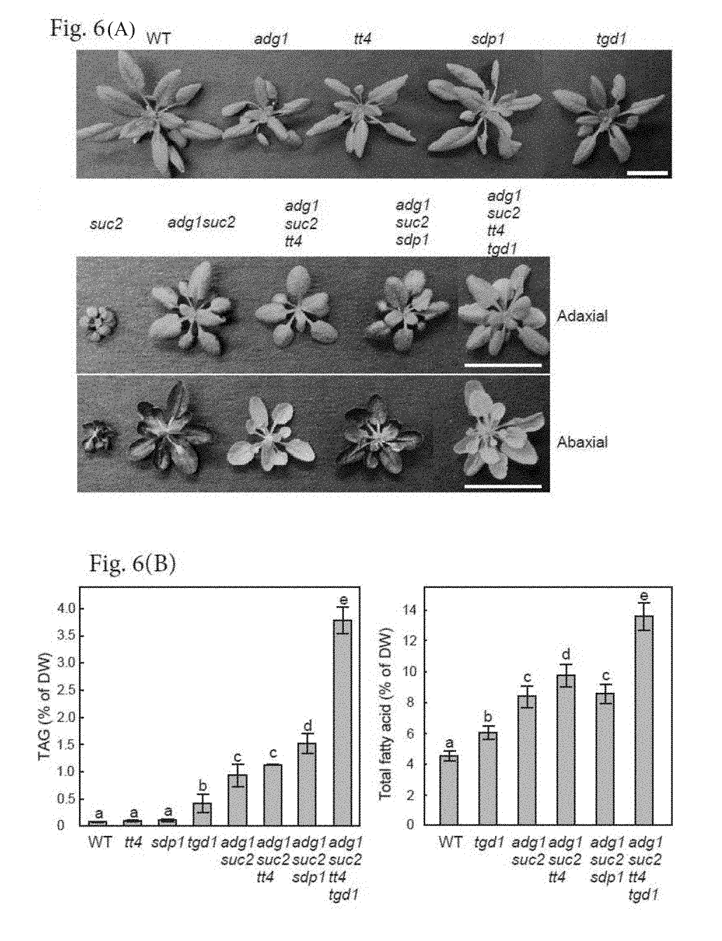

As shown in FIG. 3, adg1suc2 had upregulated gene expression of TT4 and accumulated 1.5% (of DW) of anthocyanins. TT4 catalyzed the conversion of 4-coumaroyl CoA and malonyl-CoA to naringenin chalcone in the cytosol. Malonyl-CoA may have played a role in chain elongation in FA biosynthesis in chloroplast. A test was performed to determine whether blocking anthocyanin synthesis by combining a TT4 mutant with adg1suc2 would result in an increase in FAs synthesis. To achieve this, adg1suc2 was crossed with tt4 to generate an adg1suc2tt4 triple mutant, which lost the reddish hue associated with anthocyanin accumulation (FIG. 6A). The adg1suc2tt4 triple mutant increased its total FA accumulation from 8% to 10% relative to the adg1suc2 double mutant (FIG. 6C), and a similar result was observed for TAG accumulation (FIG. 6B).

Next a test was performed to determine whether suppressing TAG turnover by introducing a mutation in the SDP1 TAG lipase would further enhance TAG accumulation in the leaves of adg1suc2. The triple adg1suc2sdp1 mutant shown in FIG. 6A was generated by crossing adg1suc2 with sdp1. TAG and total lipid quantification showed adg1suc2sdp1 increased TAG content by 66% over that accumulated by adg1suc2 while total FA content in the triple mutant was not different from that of adg1suc2 (FIG. 6B).

In this study, leaves of a 5-week-old tgd1 mutant accumulated TAG to approximately 0.4% of leaf dry weight. To determine whether the TGD1 mutation would provide a synergistic effect in the context of adg1suc2tt4 triple mutant background, the adg1suc2tt4tgd1 quadruple mutant was generated. Growth and development of the adg1suc2tt4tgd1 quadruple mutant was found to mirror that of adg1suc2 and adg1suc2tt4 (FIG. 6A). The adg1suc2tt4tgd1 quadruple mutant accumulated 3.8% TAG in 5-week-old leaves, which represents an increase of 3.4- and 50-fold over that of the adg1suc2tt4 triple mutant and wild type leaves, respectively. The levels of total FA in leaves of adg1suc2tt4tgd1 reached 13.5% of dry weight (FIG. 6B). That leaf TAG content in adg1suc2tt4tgd1 of 3.8% is much higher than that of the combined TAG of tgd1 plus adg1suc2tt4 at 1.5% (i.e., 0.4%+1.1%, respectively) indicates a positive synergistic effect TAG synthesis for this combination of mutations.

Ectopic Expression of WRI1, DGAT1 and Oleosin1 in the adg1suc2 Double Mutant Enhances TAG and Total FA Accumulation

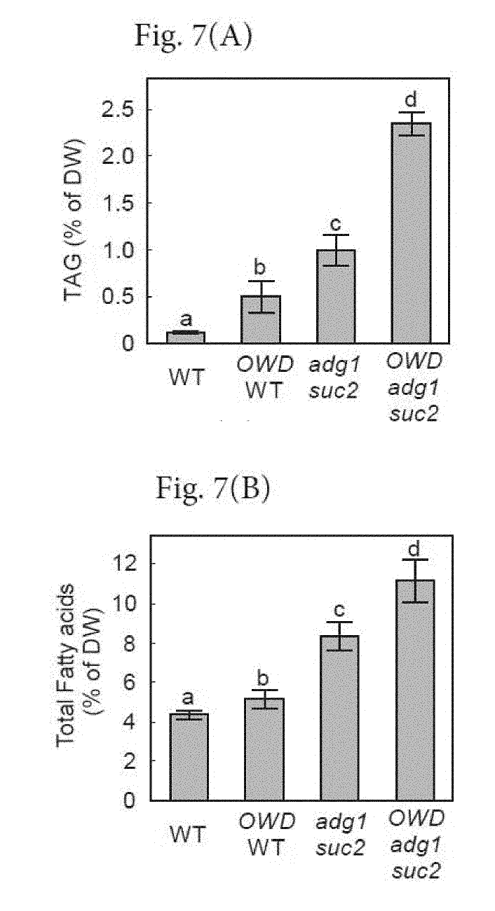

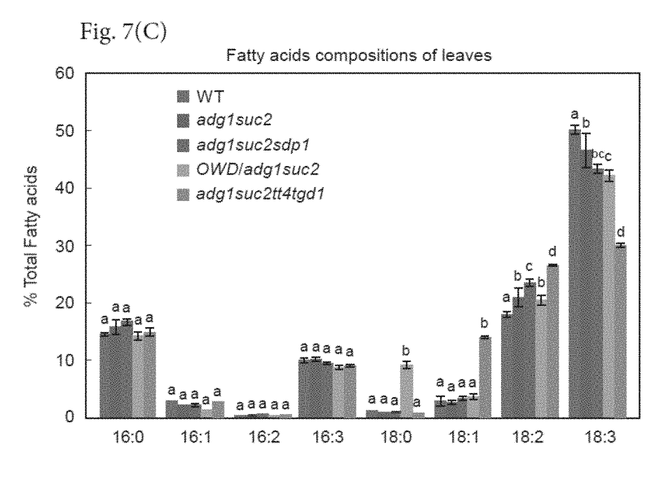

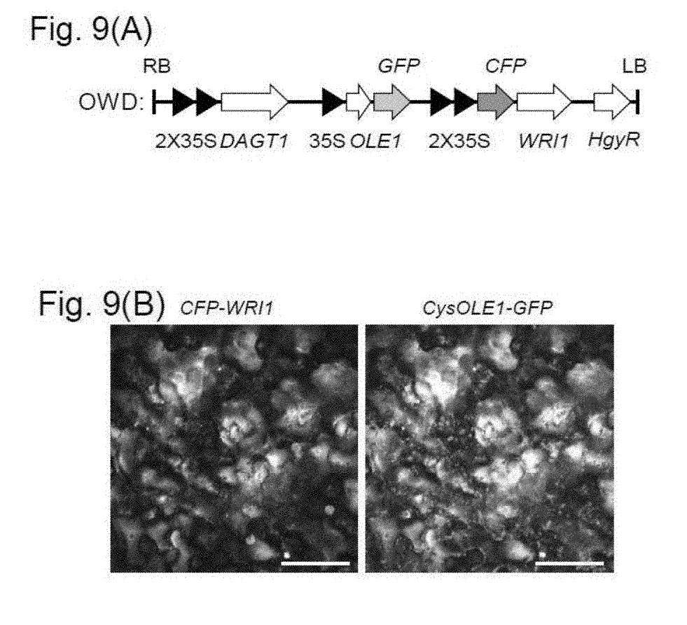

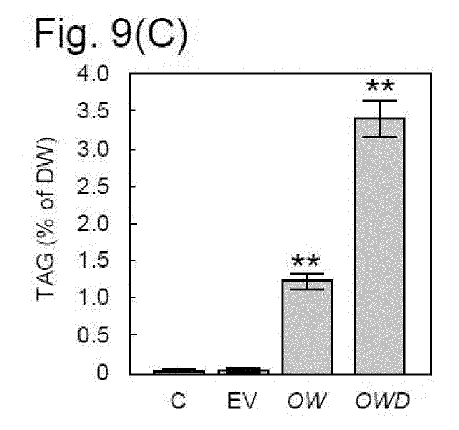

After finding that elevated leaf sugar levels could correlate with increased accumulation of both FA and TAG, the effect was explored of overexpressing several genes that have been shown to enhance lipid accumulation in tobacco. Specifically the question addressed was whether a high sugar background would further enhance the lipogenic effects of overexpressing OWD relative to their expression in wild type leaves. The OWD construct (described above) was first tested in tobacco leaves. WRI and Cys-OLE showed strong expression after two-day infiltration as visualized by fluorescence signal localized in nucleus and oil drops, respectively (FIG. 9B). After 4 days of infiltration, TAG accumulation of OWD-infiltrated leaves reached 3.3% of dry weight representing a 60-fold increase compared with leaves infiltrated with an empty vector control (FIG. 9C). Having confirmed its functionality, OWD was transformed into wild-type plants (WT). Gene transformation efficiency of WT of the OWD construct was lower than for other constructs. 3 independent transgenic lines were obtained from one transformation experiment. It was observed that ectopic overexpression of OWD resulted in a negative growth impact on WT. The biomass of the 3 OWD transgenic lines was smaller than that of WT and exhibited reduced fecundity compared with wild type plants. TAG accumulation observed in mature leaves of 5-week-old OWD transgenic plants was 0.5% (FIG. 7A). To test the hypothesis that OWD can increase TAG biosynthesis in the presence of elevated sugar, OWD/adg1suc2 transgenic plant was generated by crossing OWD/WT transgenics with the adg1suc2. TAG and total FA contents in OWD/adg1suc2 were further elevated to 2.3% and 11% of dry weight respectively, compared with 1% and 8.3% TAG and total FA contents in adg1suc2, respectively (FIG. 7B). These increases in TAG content in OWD/adg1suc2 were larger than that expected by addition of their individual effects.

The FA composition of mature leaves of WT, adg1suc2, adg1suc2sdp1, OWD/adg1suc2 and adg1suc2tt4tgd1 showed that as TAG content increased from 0.08% in WT to 3.8% in adg1suc2tt4tgd1, the levels of 18:3 declined as 18:2 became more abundant. This may suggest that the desaturation capacity for 18:2 becomes limiting as FA accumulation increases. This is consistent with previous reports that the capacity of FAD3 desaturase is limiting to the level of 18:3 accumulation. The level of 18:0 increased significantly in a FAs pool of OWD/adg1suc2 compared with adg1suc2 (FIG. 7C).

Sequence data can be found in The Arabidopsis Information Resource or GenBank database under the following accession numbers: SUC2 (AT1G22710), ADG1 (AT5G48300), SDP1 (AT5G04040), TGD1 (AT1G19800), DGAT1 (AT2G19450), TT4 (AT5G13930), TPS5 (AT4G17770), PDAT1 (AT5G13640), TMT1 (AT1G20840), TMT2 (AT4G35300), SUC4 (AT1G09960), BCCP2 (AT5G15530), KAS1 (AT5G46290), PKPβ1 (AT5G52920), OLE1 (AT4G25140), and WRI1 (AT3G54320).

### Some Embodiments

Technologies are described for methods and systems effective to increase sugar concentration in a plant, specifically leaf tissue. Mutants of Arabidopsis and expression of oleogenic factors have been used to investigate relationships among sugar availability, lipid synthesis and the accumulation of triacylglycerols (TAG) in leaf tissue. An adg1 mutation may disable a small subunit of ADP-glucose pyrophosphorylase, the first step in starch synthesis, and a suc2 mutation may disable a sucrose/proton symporter that may facilitate sucrose loading from leaves into phloem. The adg1suc2 double mutant may increase glucose plus sucrose content in leaves 80-fold relative to wild type, total fatty acid (FA) content 1.8-fold to 8.3% of dry weight (DW), and TAG more than 10-fold to 1.2% DW. The WRINKLED1 (WRI1) transcription factor also may accumulate to higher levels in leaves and the rate of FA synthesis may increase by 58%. Adding a tgd1 mutation which may disable an importer of lipids into plastids to create adg1suc2tt4tgd1, may increase total leaf FA to 13.5% DW and TAG to 3.8% DW, and a combination of these mutations may demonstrate a synergistic effect. Adding a sdp1 mutation, which may reduce degradation of TAG by the TAG lipase, may increase the TAG content by 66% to 2.0% DW and may not exhibit an effect on total FA content. Expression of the oleogenic factors WRI1 transcription factor, DGAT1 (diacylglycerol acyltransferase 1), and OLEOSIN1 oil body-associated protein in the adg1suc2 mutant may double leaf FA accumulation and may also increase TAG content to 2.3% DW. This may be a synergistic effect 4.6-fold that of the same factors expressed in wild type.

A quadruple mutant, adg1suc2tt4tgd1, results in a doubling of triacylglycerol (TAG) accumulation relative to a triple mutant (adg1suc2tt4) to 3.8% DW (dry weight). Repressing leaf sugar export and starch synthesis increases intracellular sugar concentration which potentiates fatty acid synthesis and TAG accumulation by stabilizing WRINKLED1 (WRI1) and increasing the rate of fatty acid synthesis.

It has been found with the present method that an 80-fold increase of sugars in mature, 5-week, Arabidopsis leaves as compared to wild type (WT) by decreasing sucrose export and blocking starch synthesis simultaneously in the adg1suc2 double mutant. The adg1suc2 double mutant accumulates more than 80-fold higher leaf glucose plus sucrose and approximately 10-fold more TAG than WT leaves that may be associated with an increase in accumulation of WRINKLED1 transcription factor. A series of Arabidopsis lines with increased leaf sugar accumulation were created and analyzed for their amount of total FAs and TAG. Increased levels of sugar may correlate with increased accumulation of total FA and TAG along with increased rates of FA synthesis. This may be due to a combination of two mechanisms, 1) increasing the supply of carbon skeletons, ATP and reductant and 2) inhibiting the KIN10-dependent degradation of WRI1.

The adg1suc2sdp1 triple mutant, defective in starch synthesis, may further increase TAG accumulation, and the quadruple mutant adg1suc2sdp1tgd1, deficient in plastidial lipid reimport may result in more than a doubling of TAG accumulation relative to adg1suc2sdp1. That the total FA in adg1suc2tt4tgd1accumulated to 14% of dry weight while the TAG levels were only approximately 4% may imply a bottleneck in TAG assembly.

The adg1suc2tt4 triple mutant, defective in starch and flavonoid synthesis, may further increase TAG accumulation, and the quadruple mutant adg1suc2tt4tgd1, deficient in plastidial lipid reimport may result in more than a doubling of TAG accumulation relative to adg1suc2tt4. That the total FA in adg1suc2tt4tgd1accumulated to 14% of dry weight while the TAG levels were only approximately 4% may imply a bottleneck in TAG assembly.

Overexpression of WRI1, DGAT1 and OLE1 in the adg1suc2 background may synergistically increase levels of TAG compared to the sum of their individual contributions. This may provide insight into a relationship between sugar, FA and TAG accumulation in vegetative tissues that may contribute to biotechnological efforts in optimizing TAG accumulation in vegetative tissues of plants of interest.

The relationship among sugar availability, lipid synthesis and the accumulation of triacylglycerols (TAGs) in leaf tissue can be investigated by crossing mutants in suc2 (SUC2 encoding a sucrose/H⁺ symporter that facilitates sucrose loading into phloem) and adg1 (ADG1 encoding the small subunit of ADP-glucose pyrophosphorylase, the first committed step in starch synthesis) to create a high-leaf-sugar content (e.g., greater than 10 nmole per mg fresh weight (corresponding to approximately 10-fold higher than that of WT) Arabidopsis line adg1suc2.

The leaf glucose plus sucrose content of adg1suc2 may be 80-fold higher than wild type and the total fatty acid (FA) content may be increased to 8.3% of dry weight (DW), i.e., 1.8-fold higher than that of wild type. In leaves from plants grown and matured for 5 weeks, adg1suc2 may accumulate TAG to more than 10-fold that of wild type, i.e., to 1.2% (DW). The WRINKLED1 (WRI1) transcription factor may accumulate to higher levels in adg1suc2 leaves relative to wild type and the rate of FA synthesis may increase by 58%. Reducing plastidial lipid reimport by combining adg1suc2 with tgd1 (encoding a subunit of the TGD plastid lipid reimporter) may increase total FA and TAG accumulation to 13.5% and 3.8%, respectively. The total FA in this line accumulated to 13.5% (DW) may point to a bottleneck in the assembly of TAG from FA. The effect of minimizing TAG degradation may be tested by combining adg1suc2 with sdp1 (SDP1 encoding the predominant TAG lipase) in which total FA content was similar to that of adg1suc2, but the TAG content increased by 66%.

The expression of oleogenic factors: WRI1, DGAT1 (Diacyglycerol acyltransferase1) and OLEOSIN1 (an oil body-associated protein, OLE1) in adg1suc2 doubled leaf FA accumulation and increased TAG content by 4.6-fold relative to their expression in wild type. Overexpression of WRI1, DGAT1 and OLE1 in the adg1suc2 background may lead to higher levels of TAG than expression in wild type plants.

The availability of various Arabidopsis mutant backgrounds may be utilized to probe the consequences of elevated levels of sugar on TAG accumulation in leaves by analyzing the total FA and TAG levels in various single- and multiple mutants with an initial focus on suc2, adg1 that may be combined to create a double adg1suc2 mutant.

A method for increasing oil content of vegetative tissue of a plant comprising providing the plant comprising a quadruple mutant adg1suc2tt4tgd1 or adg1suc2tt4tgd5. The method further comprising growing the plant until oil accumulates in vegetative tissue of the plant. The method 1 further comprising extracting oil from the plant. The method wherein the plant comprises seeds, leaves, stems or roots, and oil is extracted from one or more of the seeds, leaves, stems or roots. The method wherein the plant comprises leaves, and oil accumulates in the leaves at levels more than double that of a adg1suc2sdp1 triple mutant to 3.8% dry weight. The method wherein the oil is triacylglycerol (TAG).

A method for enhancing the caloric content of vegetative tissues of a plant comprising providing the plant comprising a quadruple mutant adg1suc2tt4tgd1 and growing the plant until oil accumulates in vegetative tissues of the plant.

A method for increasing oil content of leaves of a plant matured for at least 5 weeks comprising providing the plant comprising a double mutant adg1suc2 that results in an increase of sugar in the leaves at a level at least 80-fold more than that of wild type, a decrease in sucrose export and simultaneously in blocking starch synthesis. The method further comprising growing the plant until oil accumulates in vegetative tissue of the plant. The method further comprising extracting oil from the plant. The method wherein the plant comprises seeds, leaves, stems or roots, and oil is extracted from one or more of the seeds, leaves, stems or roots. The method wherein the plant accumulates oil at levels at least 10 times more than wild type. The method wherein the plant comprises leaves, and oil accumulates in the leaves at levels at least 10-fold more than that of wild type leaves. The method wherein the plant comprises leaves, and oil accumulates in the leaves at levels at least 10-fold more than that of wild type leaves associated with an increase in accumulation of WRINKLED1 transcription factor. The method wherein the oil is triacylglycerol (TAG). The method wherein the plant is Arabidopsis.

A method for increasing total fatty acid and triacylglycerol content of vegetative tissue of a plant comprising providing the plant comprising a quadruple mutant adg1suc2tt4tgd1 that results in increased levels of sugar coupled with an inhibition of starch synthesis that results in an increased rate of fatty acid synthesis. The method wherein the plant is Arabidopsis and the vegetative tissue is leaves.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### INCORPORATION OF SEQUENCE LISTING

Incorporated herein by reference in its entirety is the Sequence Listing for the application. The Sequence Listing is disclosed on a computer-readable ASCII text file titled, “BSA17-08 IP2017-011-02 substitute sequence listing.txt”, created on Jan. 25, 2019. The sequence listing.txt file is 8000 bytes in size.

1. A plant, or a vegetative plant tissue thereof, comprising two mutations, wherein the two mutations are an adg1 mutation and a suc2 mutation, wherein leaves of the plant have at least about 50-fold more sugar than a counterpart wild type plant.

2. The plant of claim 1, wherein the plant is from the Brassicaceae family or Solanaceae family.

3. The plant of claim 1A, wherein the plant is selected from the following species Camelina sativa, Thlaspi arvense, Brassica napus, Arabidopsis thaliana, Nicotiana tobaccum and Nicotiana benthamiana.

4. A method of increasing oil content in the vegetative tissue of a plant, the method comprising providing the plant with two mutations, wherein the two mutations are an adg1 mutation and a suc2 mutation.

5. The method of claim 4, wherein the total fatty acid content in the vegetative tissue of the plant is at least about 100% greater than that of vegetative tissue of a counterpart wild type plant.

6. A plant, or a vegetative plant tissue thereof, comprising three mutations, wherein the three mutations are:
an adg1 mutation, a suc2 mutation and a sdp1 mutation, or
an adg1 mutation, a suc2 mutation and a tt4 mutation,
wherein leaves of the plant have at least about 25% higher triacylglycerol content than a counterpart plant comprising an adg1 mutation and a suc2 mutation.

7. The plant of claim 6, wherein the plant is from the Brassicaceae family or Solanaceae family.

8. The plant of claim 7, wherein the plant is selected from the following species Camelina sativa, Thlaspi arvense, Brassica napus, Arabidopsis thaliana, Nicotiana tobaccum and Nicotiana benthamiana.

9. A method of increasing oil content in the vegetative tissue of a plant, the method comprising providing the plant with three mutations, wherein the three mutations are:
an adg1 mutation, a suc2 mutation and a sdp1 mutation, or
an adg1 mutation, a suc2 mutation and a tt4 mutation.

10. The method of claim 9, wherein the triacylglycerol content in the vegetative tissue of the plant is at least about 25% greater than that of vegetative tissue of a counterpart plant comprising two mutations wherein the two mutations are an adg1 mutation and a suc2 mutation.

11. A plant, or a vegetative plant tissue thereof, comprising four mutations, wherein the four mutations are:
an adg1 mutation, a suc2 mutation, an sdp1 mutation, and a tgd1 mutation, or
an adg1 mutation, a suc2 mutation, a tt4 mutation and a tgd1 mutation,
wherein leaves of the plant have at least about 10 times greater triacylglycerol content than a counterpart wild type plant.

12. The plant of claim 11, wherein the plant is from the Brassicaceae family or Solanaceae family.

13. The plant of claim 12, wherein the plant is selected from the following species Camelina sativa, Thlaspi arvense, Brassica napus, Arabidopsis thaliana, Nicotiana tobaccum and Nicotiana benthamiana.

14. A method of increasing oil content in the vegetative tissue of a plant, the method comprising providing the plant with four mutations, wherein the four mutations are:
an adg1 mutation, a suc2 mutation, an sdp1 mutation, and a tgd1 mutation, or
an adg1 mutation, a suc2 mutation, a tt4 mutation and a tgd1 mutation.

15. The method of claim 14, wherein the triacylglycerol content in the vegetative tissue of the plant is at least about 10 times greater than that of vegetative tissue of a counterpart wild type plant.
